(19) United States
(12) Patent Application Publication
(10) Pub. No.: US 2026/0166331 A1
(43) Pub. Date: Jun. 18, 2026

(54) FLEXIBLE PHOTOTHERAPY DEVICE

(71) Applicant: Shenzhen Kaiyan Medical Equipment Co., Ltd., Shenzhen (CN)

(72) Inventors: Alain Dijkstra, Amstelveen (NL); Hu Chunlei, Shenzhen (CN); Yan Yuansong, Shenzhen (CN)

(73) Assignee: Shenzhen Kaiyan Medical Equipment Co., Ltd., Shenzhen (CN)

(21) Appl. No.: 19/419,418

(22) Filed: Dec. 15, 2025

(30) Foreign Application Priority Data
Dec. 17, 2024 (CN) 2024231233168

(51) Int. Cl. A61N 5/06

(52) U.S. Cl. CPC A61N 5/0616; A61N 2005/0627; A61N 2005/0634; A61N 2005/0642; A61N 2005/0651; A61N 2005/0664

(57) ABSTRACT

Embodiments of the present invention disclose a flexible phototherapy device comprising a storage seat with a storage cavity and a flexible light board that automatically retracts around a rotating shaft driven by a driving member. A limiting member at the distal end prevents over-retraction, while flexible wear-resistant strips protect the board edges. The flexible light board includes flexible light strips arranged in transverse and longitudinal patterns for uniform illumination. A conductive interface with conductive rings and spring pins maintains electrical connectivity during rotation. An integrated solar panel recharges an onboard energy storage element for cordless operation. An IR sensor detects user presence to automatically activate or deactivate the LEDs. In an alternative configuration, multiple flexible light boards slide horizontally along a top bar via a motion conversion mechanism for window coverage applications.

### TECHNICAL FIELD

The present invention relates to the field of personal care and therapeutic devices, more particularly to a flexible phototherapy device, configured to provide phototherapy, designed for therapeutic use, combining light therapy with mechanical and electrical features to improve portability and convenience.

### BACKGROUND ART

Phototherapy has become increasingly utilized in cosmetic, dermatological, and wellness applications for delivering therapeutic light energy to targeted areas of the body. Many existing phototherapy devices rely on rigid light-emitting panels or fixed structures that can limit adaptability to different body contours or restrict user mobility. To improve comfort and treatment effectiveness, various approaches have introduced flexible light-emitting substrates that can better conform to curved anatomical regions. However, such flexible structures can be difficult to manage, store, or deploy conveniently and hygienically.

Users generally prefer phototherapy products that are compact, easy to handle, and capable of being deployed quickly without complex setup. Traditional flexible phototherapy pads, once unfolded, can be cumbersome to reposition or store, often requiring manual rolling or folding that risks damaging embedded electrical components. Additionally, maintaining a reliable electrical connection and controlled illumination during the movement or adjustment of flexible light elements can pose challenges.

Accordingly, there remains a need for improved devices, systems, and methods that incorporate flexible light-emitting structures while providing convenient deployment, retraction, organization, and power delivery. There is also a need for solutions that enhance durability and user experience while enabling efficient storage and controlled operation of flexible phototherapy elements.

### OBJECTS OF THE INVENTION

Some of the objects of the invention are as follows:

An object of the present invention is to provide a flexible phototherapy device featuring a flexible light board that can be wound automatically into an internal storage cavity, thereby reducing the device's storage volume and enhancing portability.

A further object of the present invention is to integrate a driving mechanism, a small motor, and a rotating shaft assembly inside a storage seat that rotates to roll or unroll the flexible light board, allowing the pad to be deployed or retracted with minimal manual effort.

Another object of the present invention is to include a limiting end member at the free end of the flexible light board, having a transverse dimension larger than a storage port, so that when the board is fully retracted, it abuts the port opening, absorbing terminal impacts on the storage seat.

A further object of the present invention is to incorporate flexible wear-resistant strips along the side edges of the flexible light board, contacting the edges of the storage port during winding, shielding the board's edge from friction and extending its service life, and stiffening the board to maintain its shape when deployed.

Yet another object of the present invention is to embed the LED light source within a flexible pad. One end of the light strip is secured to the rotating shaft, and the light strip runs inside a silicone or polymer pad.

A further object of the present invention is to arrange multiple flexible light strips on the flexible pad for expanded coverage. Specifically, a first light strip extends across the width of the flexible pad, several second strips extend lengthwise away from the rotating shaft, distributing flexible light strips in a grid-like pattern, uniform irradiation, and improving treatment efficacy.

Another object of the present invention is to provide an on-board energy solution, such as a rechargeable energy storage element, coupled with an integrated solar panel. Ambient light charges the battery through the device's circuitry, so that the flexible pad can be operated cordless and recharged anywhere, even without an external outlet.

A further object of the present invention is to ensure reliable electrical contact to the rotating end of the light board. To this end, the invention employs a stationary circuit board and a conductive spring-pin assembly, maintaining power transfer to the first end of the flexible light board during rotation, preventing disconnection due to movement.

A further object of the present invention is to construct the device compactly, with the drive motor placed axially inside the rotating shaft, minimizing extra space. Coaxial components of the rotating shaft, conductive disk, and support block are aligned to save volume.

### SUMMARY OF THE INVENTION

According to a first aspect of the present invention, a flexible phototherapy device is provided. The flexible phototherapy device comprises: a storage seat defining a storage cavity and a storage port communicating with the storage cavity; a flexible light board having a proximal end and a distal end, the proximal end being fixed to a rotating shaft that is rotatably supported within the storage cavity, the distal end being extendable through the storage port; a driving member operatively coupled to the rotating shaft and configured to rotate the rotating shaft to wind the flexible light board around the rotating shaft for retraction of the flexible light board into the storage cavity; and a conductive interface located in the storage cavity and electrically connected to the proximal end of the flexible light board to provide electrical power to the flexible light board during extension and retraction.

In one embodiment of the invention, the distal end of the flexible light board includes a limiting member having a width greater than the width of the storage port, the limiting member preventing full retraction of the distal end into the storage cavity.

In one embodiment of the invention, a flexible phototherapy device further comprising a flexible wear-resistant strip disposed on at least one side of the flexible light board and configured to reduce abrasion between the flexible light board and the storage port.

In one embodiment of the invention, the flexible light board comprises a flexible pad and a flexible light strip embedded in the flexible pad.

In one embodiment of the invention, the flexible light strip comprises a transverse light strip extending across a width of the flexible pad and multiple longitudinal light strips connected to the transverse light strip and extending along a length of the flexible pad.

In one embodiment of the invention, the conductive interface comprises a fixed conductive member electrically connected to a circuit board in the storage cavity and a movable conductive member coupled to the rotating shaft.

In one embodiment of the invention, the fixed conductive member comprises a conductive ring and the movable conductive member comprises a spring-biased conductive spring pin in sliding contact with the conductive ring.

In one embodiment of the invention, the drive member has a rotatable output shaft extending into the interior of the rotating shaft and coupled thereto.

In one embodiment of the invention, the flexible phototherapy device further comprises a transmission block secured to the rotatable output shaft and positioned inside the rotating shaft to transmit rotational force to the rotating shaft.

In one embodiment of the invention, the storage seat comprises a first housing and a second housing portion together along a seam, the first housing including a connection hole aligned with a connection hole of the second housing, and an insertion structure extending into the seam at an angle between 60° and 90° relative to an axis of the connection holes.

According to a second aspect of the present invention, a method for deploying and retracting a flexible phototherapy light board is provided. The method comprising: coupling a proximal end of a flexible light board to a rotating shaft positioned within a storage seat; rotating the rotating shaft in a first direction with a drive member to wind the flexible light board around the rotating shaft and retract the flexible light board into a storage cavity of the storage seat; and extending the flexible light board from the storage cavity by rotating the rotating shaft in a second, opposite direction or by pulling the flexible light board outward through a storage port.

In one embodiment of the invention, rotating the rotating shaft in the first direction comprises driving a rotatable output shaft disposed within the rotating shaft.

In one embodiment of the invention, the method further comprising maintaining electrical conductivity between the flexible light board and a circuit board through a conductive interface while the rotating shaft rotates.

In one embodiment of the invention, the method further comprising preventing complete retraction of the flexible light board by engaging a limiting member at its distal end with an edge of the storage port.

According to a third aspect of the invention, a phototherapy system is provided. The phototherapy system comprising: a storage seat defining a storage cavity and a storage port communicating with the storage cavity; a flexible light board having a proximal end and a distal end, the proximal end being fixed to a rotating shaft that is rotatably supported within the storage cavity, the distal end being extendable through the storage port; a drive member operatively coupled to the rotating shaft and configured to rotate the rotating shaft to wind the flexible light board around the rotating shaft for retraction of the flexible light board into the storage cavity; and a conductive interface located in the storage cavity and electrically connected to the proximal end of the flexible light board to provide electrical power to the flexible light board during extension and retraction; and a control module configured to operate the drive member to retract and deploy the flexible light board and to supply power to the flexible light board through the conductive interface.

In one embodiment of the invention, the control module provides at least a retract mode, a deploy mode, and an illumination mode.

In one embodiment of the invention, the phototherapy system further comprising at least one sensor in the storage cavity configured to monitor temperature or electrical contact quality, and the control module adjusts the operation of the drive member based on sensor input.

In one embodiment of the invention, a sealing strip is positioned between the housing portions of the storage seat to inhibit ingress of dust or moisture.

In one embodiment of the invention, the flexible light board includes flexible wear-resistant strips on opposite sides extending from the proximal end to the distal end.

In one embodiment of the invention, the rotating shaft includes a positioning feature, and a transmission block or rotating disk includes a complementary engagement feature to establish a positive rotational coupling.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, the term “flexible phototherapy device” refers to any device configured to emit therapeutic light for skin treatment, pain relief, or wellness applications.

In the context of the specification, the term “phototherapy element” encompasses any light-emitting device capable of emitting light of therapeutic wavelength(s), including but not limited to light-emitting diodes (LEDs), organic LEDs (OLEDs), laser diodes, or equivalent optical sources. The light may include ultraviolet, visible, near-infrared, or far-infrared spectra.

In the context of the specification, the term “storage seat” or “housing” is intended to cover any casing, enclosure, or structural body that contains or supports components of the device. The housing may include a handle portion, head portion, or other segments, and may be made from polymeric, metallic, composite, or other suitable materials.

In the context of the specification, the terms “flexible light board” or “flexible light strip” refer to a portion of the device coupled to the storage seat and configured to emit light toward the skin. The head may include one or more light-transmitting surfaces, optical lenses, or diffusers, and may also support electrodes or other stimulation elements.

In the context of the specification, the term “control interface” refers to any input or output mechanism enabling a user to operate the device. The control interface may include physical buttons, capacitive touch sensors, sliders, switches, or graphical displays, and may further include wireless control via a mobile application.

In the context of the specification, the term “circuit board” encompasses any printed circuit board (PCB), flexible circuit, or equivalent substrate that supports and electrically connects components of the device, including power supplies, control chips, drivers, or stimulation elements.

In the context of the specification, the term “user” or “subject” is intended to broadly cover humans, animals, or other recipients of the treatment, unless otherwise specifically limited.

In the context of the specification, the term “LED module” refers to one or more light-emitting diode (LED) elements that are electrically connected and configured to emit light of specific wavelengths suitable for therapeutic purposes. The LED module may include drive circuitry, heat dissipation structures, and optical elements such as lenses or diffusers to control light distribution.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, or Super luminous diodes (“SLD”).

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelengths, wherein the range is inclusive of visible light, UV and IR frequencies and wavelengths. It is to be noted here that UV radiation can be categorized in several ways depending on respective wavelength ranges, all of which are envisaged to be under the scope of this invention. For example, UV radiation can be categorized as Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm). Similarly, IR radiation may also be categorized into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm), and Far IR (15-1000 μm).

Unless otherwise stated, the term “light” as used in this specification encompasses electromagnetic radiation in the visible (380-780 nm) and infrared (780 nm-1000 nm) ranges, particularly red light (620-750 nm) and near-infrared (750-1400 nm) wavelengths commonly used in photobiomodulation therapy. Particular wavelengths which may be selected as the dominant emissive wavelength may include the follow, without any preference to be indicated by order: 400 nm, 405 nm, 420 nm, 430 nm, 450 nm, 465 nm, 515 nm, 530 nm, 532 nm, 590 nm, 630 nm, 633 nm, 640 nm, 650 nm, 655 nm, 660 nm, 670 nm, 680 nm, 780 nm, 785 nm, 810 nm, 830 nm, 840 nm, 850 nm, 860 nm, 870 nm, 904 nm, 915 nm, 980 nm, 1015 nm, 1060 nm, 1065 nm, 1070 nm, 1200, and 1400 nm. As used herein, the term “light therapy” refers to the use of one or more light sources of any type that emit light with a wavelength between about 400 and 1400 nm. The device may also emit blue or ultraviolet light for surface-level treatments such as acne reduction or microbial control.

The red light (approximately 630-660 nm) penetrates deeply into the scalp to stimulate blood circulation and enhance hair follicle activity, thus promoting hair growth and repair. Blue light (around 415-470 nm) exhibits antibacterial properties and is effective in treating scalp acne and reducing inflammation. Green light (approximately 520-540 nm) can help reduce pigmentation and soothe sensitive or irritated scalp tissue. Yellow light (around 580-600 nm) improves oxygen exchange in the cells and aids in detoxifying the scalp, while near-infrared light (800-850 nm) reaches deeper layers to accelerate healing and reduce pain and inflammation. Green light (approximately 520-540 nm) can help reduce pigmentation and soothe sensitive or irritated scalp tissue. Yellow light (around 580-600 nm) improves oxygen exchange in the cells and aids in detoxifying the scalp, while near-infrared light (800-850 nm) reaches deeper layers to accelerate healing and reduce pain.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 shows a flexible phototherapy device, in accordance with an embodiment of the present invention.

FIG. 2 is an exploded view showing the flexible phototherapy device, storage seat, flexible light board, solar panel, and limiting member, in accordance with an embodiment of the present invention.

FIG. 3 shows an exploded view of the top portion of the flexible phototherapy device, in accordance with an embodiment of the present invention.

FIG. 4 is an enlarged view of the flexible phototherapy device, showing various components, in accordance with an embodiment of the present invention.

FIG. 5 shows a side perspective view of the flexible phototherapy device, in accordance with an embodiment of the present invention.

FIG. 6 shows the flexible phototherapy device configured with a horizontal sliding channel arrangement, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

Embodiments of the present invention disclose a flexible phototherapy device. The features described in connection with any embodiment may be combined with other embodiments unless context indicates otherwise.

In an embodiment, the flexible phototherapy device comprises a storage seat with an internal cavity and a storage port or opening to the outside. Inside the cavity, a rotating shaft is mounted on bearings. A flexible light board is attached to the rotating shaft; its proximal end is fixed to the rotating shaft, and its distal end extends out through the storage port when the device is unrolled. The flexible light board also includes a driving member inside the storage seat that drives the rotating shaft to wind or unwind the board. In this way, when the flexible light board is being stored, the motor turns the rotating shaft to automatically roll the flexible light board into the cavity through the storage port. During deployment, the flexible light board is unwound either by reversing the motor or by manually pulling the board outward. This self-retracting mechanism reduces the overall size of the flexible light board when stored and protects most of the light board inside the housing.

In use, when winding the flexible light board into the storage seat, the outer portion of the flexible light board is drawn through the storage port and wraps around the rotating shaft. The limiting member attached to the board's distal end stops at the port edge, ensuring that winding halts at the correct point. The motorized winding eliminates the need for the user to manually coil the board, thus simplifying storage and avoiding tangles. Because most of the board is enclosed during storage, its exposure to mechanical damage is greatly reduced.

In some embodiments, the flexible light board includes a limiting member at the distal end of the flexible light board. This limiting member is wider than the storage port, so it cannot pass inside the housing. During winding, the limiting member eventually comes to rest against the edge of the port. At this point, the entire flexible light board is fully retracted, and the limiting member braces against the port edge. This construction prevents the flexible light board from being pulled completely inside, making it easy to grab by the limiting member and pull the flexible light board back out again when deploying. Also, if the housing is ever impacted or compressed during transport, the limiting member transmits the force to the housing edge rather than to the fragile board material, thus protecting the flexible light strips from shock. This edge-stop design is analogous to known support structures that are made wider than the flexible component to act as impact guards.

In some embodiments, along each side of the flexible light board, flexible wear-resistant strips are provided. These flexible light strips are made of a durable rubber or fabric material. They are placed between the edges of the flexible light board and the sides of the storage port. When the board is wound or unwound, the flexible light strips, rather than the flexible light board itself, make contact with the sealing port edges. This greatly reduces friction on the flexible light board edges and prevents abrasion of the circuit board. Additionally, these flexible light strips slightly stiffen the flexible light board edges; when the board is fully unrolled, the flexible light strips help maintain its planar shape. In the stored state, the flexible light strips also prevent the board from curling excessively by distributing forces along its sides.

In some embodiments, the flexible light board comprises a flexible pad with a flexible light strip. One end of the flexible light strip is connected to the rotating shaft at the storage end and is thus held fixed when wound. The flexible light strip runs lengthwise along the flexible pad, and the flexible pad material encapsulates or covers the strip to shield it from external contact. The flexible pad includes a shallow channel or slot in which the flexible light strip sits, sidewalls, and notches in the channel that hold the flexible light strip in place and keep it from rubbing against outside objects. This arrangement protects the flexible light strip and wiring from wear or accidental touches. The use of a silicone flexible pad around the flexible light strip also insulates and cushions the flexible light strip, reducing the risk of damage if the flexible pad flexes or hits something.

To ensure a wide, uniform treatment area, the flexible light strip assembly is split into multiple sections. A transverse light strip (also referred to as first light strip) runs laterally across the width of the flexible pad near the rotating shaft. This transverse light strip connects directly to the circuit board inside the housing for power input. Along the length of the pad, there are several longitudinal light strips (also referred to as second light strips) arranged at intervals in parallel. When powered, the transverse light strip supplies illumination across the flexible pad's width and also feeds the longitudinal light strips. Such a flexible light strip array layout produces uniform lighting over the entire flexible pad surface. This approach is motivated by known techniques to improve light coverage, as flexible light strip arrays have been used to achieve more even phototherapy illumination. In practice, each of the longitudinal light strips is laid out along the length of the flexible pad and can be uniformly wound onto the rotating shaft together with the flexible pad, so that no flexible light strip is tighter or looser than any other when the flexible pad is rolled up.

In some embodiments, the flexible pad includes an integrated solar panel on the sides. The solar panel is placed on the exposed surface of the flexible pad between the second light strips and flexible wear-resistant strips. The solar panel is made of a flexible photovoltaic material so that it can curve with the flexible pad. Its purpose is to harvest ambient sunlight to recharge the flexible pad's internal energy storage element. When outdoors or near a light source, the solar panel converts light into electricity, which is routed through the circuit board to the energy storage element. Similar concepts have been applied in solar-powered flexible phototherapy devices; for example, a solar-powered foldable phototherapy unit has been demonstrated for treating neonatal jaundice. By including a solar panel in the flexible pad, the user can extend usage time without needing a plug-in power source, greatly enhancing portability.

An energy storage element, such as a rechargeable lithium-ion battery or supercapacitor, is provided in the housing to store power. The solar panel and any external power input connect to this energy storage element through a charging management circuit on the circuit board. The management circuit regulates voltage and current to safely charge the energy storage element from both the solar panel and any other source, and prevents overcharging. During therapy, the energy storage element supplies power to the flexible light strip. In this way, the device can operate for extended periods without external power. The combination of a flexible solar panel and rechargeable energy storage element allows the flexible pad to be used in field or travel situations where outlets are unavailable, without interrupting treatment.

The circuit board is mounted inside the storage housing near the rotating shaft. It handles the flexible light strip driving and charging functions. In a preferred arrangement, the second light strips on the flexible pad are directly connected to the first light strip, minimizing the number of wires and simplifying the wiring layout. This direct connection shortens the path from the flexible light strips to the power source, reducing losses and making the assembly more reliable. When the flexible pad is wound, the first light strip feeds all the others; when unrolled, the configuration ensures a uniform illumination because each second strip is driven by the same source.

To maintain electrical continuity through the winding mechanism, a conductive interface is used. The conductive interface includes a conductive disk assembly comprising a stationary mounting disk fixed to the housing, and a rotating disk fixed to the end of the shaft. These disks interface electrically via spring-loaded contacts and rings. The mounting disk carries one or more concentric conductive rings, and the rotating disk has corresponding conductive spring pins that press against these conductive rings. This slip-ring-like assembly allows the flexible light board's electrical connector at the rotating shaft end to remain powered regardless of the rotating shaft's rotation. Direct hardwired connections at a rotating joint tend to fail under stress, but a spring-pin on a conductive-ring interface accommodates motion and vibration. Thus, even as the shaft spins to wind or unwind the board, the electrical power path to the flexible light strip stays intact and reliable.

In some embodiments, additional components are provided on the rotating shaft to improve mechanical coupling. A rotation transmission block is sleeved over the motor's rotatable output shaft and loosely fits inside the rotating shaft. The block has a second positioning groove that mates with a positioning protrusion on the shaft, so they rotate together without the need for any screws. The rotation transmission block increases the contact area between the motor output and the rotating shaft, ensuring efficient torque transfer. Similarly, a rotating support block is placed between the motor and the rotating shaft ends, also keyed to the rotating shaft, to stabilize rotation and prevent wobble. These components provide axial support to the rotating shaft and the flexible pad, improving reliability. They are each aligned to the rotating shaft via mating positioning protrusions and positioning grooves.

In some embodiments, the driving member is realized by a small electric motor, a geared motor, or a rotary actuator. Notably, the motor and its rotatable output shaft are positioned coaxially inside the rotating shaft. This internal arrangement means the motor's rotating output engages the flexible pad shaft directly, eliminating additional gears or couplings and saving external space. For example, the motor's drive shaft extends into the hollow rotating shaft and attaches to the rotation transmission block. Because the motor is partially enclosed by the rotating shaft, the overall height of the device is reduced. In one implementation, the motor provides enough torque to wind the entire board automatically; in another, it provides a retracting force while the user pulls the flexible pad out by hand to unroll it.

In some embodiments, the storage seat typically consists of two parts: a first housing and a second housing that together enclose the internal components. The first housing and the second housing are made of molded plastic or metal. To join them, one of the housing include holes and the other housing has matching holes for alignment; screws or fasteners can be inserted through these holes to clamp the first housing and the second housing together. Further, a sealing gasket or strip is placed between the first housing and the second housing to keep dust and moisture out of the cavity. In other embodiments, a snap-fit insertion structure is used: one of the first or second housing has a socket and the other a corresponding insert tab that slides or clips into the socket. The plug-in or snap-fit connection allows the parts to be assembled without tools. For added security, the insert direction and fastener direction can be arranged orthogonally so that the combined connection resists loosening under vibration. Once assembled, the housing forms a robust enclosure that supports the shaft and motor and provides the finished exterior of the flexible pad.

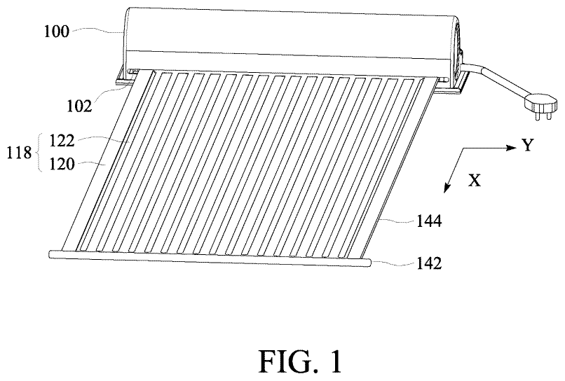

Embodiments of the present invention will now be described with reference to FIGS. 1 to 5.

Referring to FIGS. 1 to 5, the flexible phototherapy device provided in the present application includes a storage seat 100, a flexible light board 118, a rotating shaft 128, and a driving member 132. The storage seat 100 defines a storage cavity and includes a storage port 102 communicating with the storage cavity. The rotating shaft 128 is rotatably mounted within the storage cavity. A proximal end of the flexible light board 118 is connected to the rotating shaft 128, and a distal end of the flexible light board 118 extends outwardly through the storage port 102. The driving member 132 is disposed in the storage seat 100 and is configured to drive the rotating shaft 128 to rotate, thereby enabling the flexible light board 118 to be wound around the rotating shaft 128.

When the flexible pad 120 is to be stored, the driving member 132 drives the rotating shaft 128 to rotate. Because the proximal end of the flexible light board 118 is connected to the rotating shaft 128, the flexible light board 118 is guided into the storage cavity through the storage port 102 and is wound around the rotating shaft 128. This configuration enables automatic winding, reduces the storage volume of the flexible pad 120, and facilitates transportation and carrying. Because at least a major portion of the flexible light board 118 is enclosed within the storage cavity, exposure is reduced, and the likelihood of damage during transport and storage is minimized, thereby enhancing safety and reliability.

When the flexible phototherapy device is to be deployed, the driving member 132 drives the rotating shaft 128 to rotate in the reverse direction. The user can also manually pull the flexible light board 118 outward. The manner of unfolding is not limited herein.

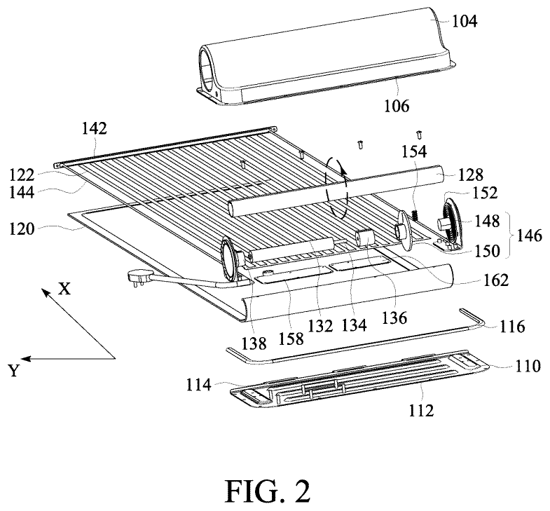

In some embodiments, and referring to FIGS. 1 and 2, the distal end of the flexible light board 118 is connected to a limiting member 142. The limiting member 142 has a width greater than the width of the storage port 102, such that the limiting member 142 cannot pass through the storage port 102 into the storage cavity. When the flexible light board is fully stored, the limiting member 142 remains positioned outside the storage cavity. Upon unfolding, the limiting member 142 can pull the flexible light board 118 smoothly outward through the storage port 102, simplifying the unfolding process.

Additionally, during transportation or storage, even if the storage seat 100 is subjected to external impact or compression, the limiting member 142 abuts the edge of the storage port 102 and prevents direct force from being transmitted to the flexible light board 118, thereby reducing damage to the flexible light board 118. The connection between the limiting member 142 and the flexible light board 118 further improves the structural stability of the overall device. The limiting member 142 also serves as a support point for the flexible light board 118, helping maintain its shape and positional stability.

In some embodiments, the width of the limiting member 142 being greater than that of the storage port 102 refers to at least one region of the limiting member 142 having a width that exceeds the corresponding width of the storage port 102 at that location, thereby ensuring that the limiting member 142 rests against the outer edge of the storage port 102 when the device is stored.

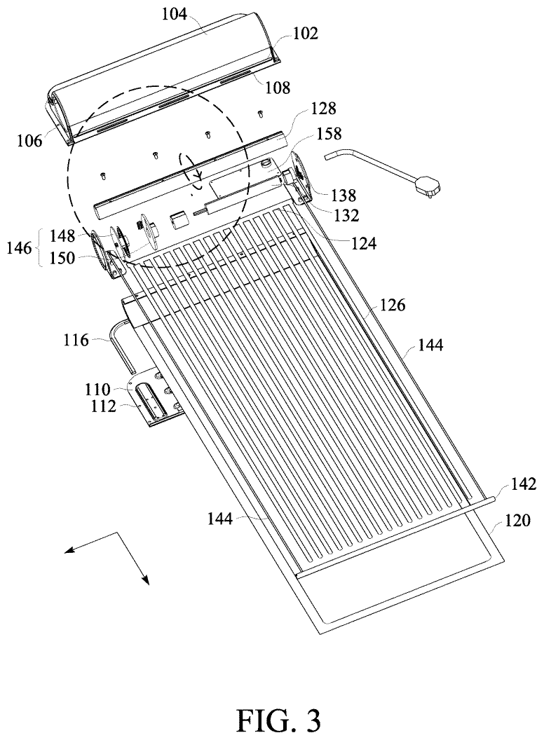

In some embodiments, and referring to FIGS. 3 to 5, at least one side of the flexible light board 118 is provided with a flexible wear-resistant strip 144. The flexible wear-resistant strip 144 is positioned between the side of the flexible light board 118 and the sidewall of the storage port 102. During winding and unwinding, the flexible wear-resistant strip 144 contacts the sidewall of the storage port 102 in place of direct contact by the flexible light board 118, thereby reducing wear and extending the service life of the flexible light board 118.

In addition, the flexible wear-resistant strip 144 reinforces the flexible light board 118 when it is in the unfolded state. The flexible wear-resistant strip 144 helps maintain the shape and positional stability of the flexible light board 118. In the stowed state, the flexible wear-resistant strip 144 helps prevent the flexible light board 118 from becoming deformed or damaged due to excessive curling. In an embodiment, the flexible wear-resistant strip 144 is formed without being restricted to one or in combination of wear-resistant rubber strips, wear-resistant fabric strips, etc.

Specifically, the flexible wear-resistant strip 144 extends along the flexible light board 118 from the proximal end of the flexible light board 118 toward the distal end, thereby maximizing coverage and providing protection along the entire side of the flexible light board 118 to enhance overall stability and safety during storage and deployment. The flexible wear-resistant strips 144 are provided on both sides of the flexible light board 118, allowing both sides of the flexible light board 118 to avoid frictional contact with the sidewalls of the storage port 102.

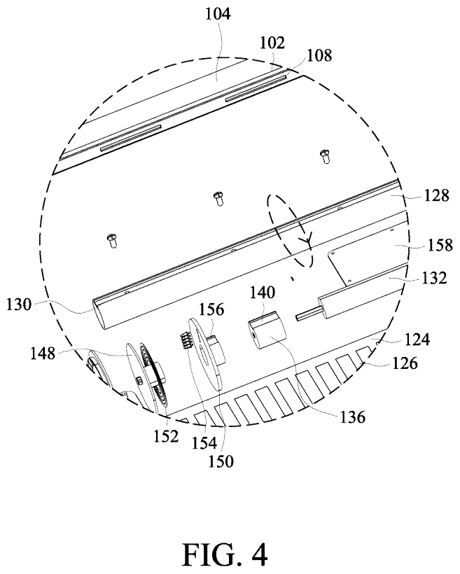

Referring to FIGS. 2 and 4, in some embodiments, the flexible light board 118 includes a flexible pad 120 and a flexible light strip 122. One end of the flexible light strip 122 is connected to the rotating shaft 128, and the flexible light strip 122 is mounted within the flexible pad 120. The flexible pad 120 protects the flexible light strip 122, reducing wear and damage caused by friction, collision, or other external factors. In an embodiment, the flexible light strip 122 is embedded within the flexible pad 120 to avoid exposure and further protect the flexible light strip 122. The flexible pad 120 includes a mounting slot, and the flexible light strip 122 is received within the mounting slot, facilitating installation and maintenance. The sidewalls and notches of the mounting slot isolate the flexible light strip 122 from external friction, thereby improving the protection of the flexible light strip 122.

Referring to FIGS. 3 and 4, the flexible light strip 122 includes a transverse light strip 124 (also referred to as first light strip) and a plurality of longitudinal light strips 126 (also referred to as second light strips). The transverse light strip 124 extends along the width direction or Y-direction of the flexible pad 120, which facilitates uniform winding of the transverse light strip 124 and the flexible pad 120 onto the rotating shaft 128. The transverse light strip 124 is located at the end of the flexible pad 120 closest to the rotating shaft 128, and the transverse light strip 124 is connected to a circuit board 158 located near the storage cavity. Along the width direction (Y) of the flexible pad 120, the transverse light strip 124 and the longitudinal light strips 126 are connected at intervals. By powering the transverse light strip 124, a longer illumination length is achieved, which allows additional longitudinal light strips 126 to be incorporated, thereby increasing the number of phototherapy irradiation areas.

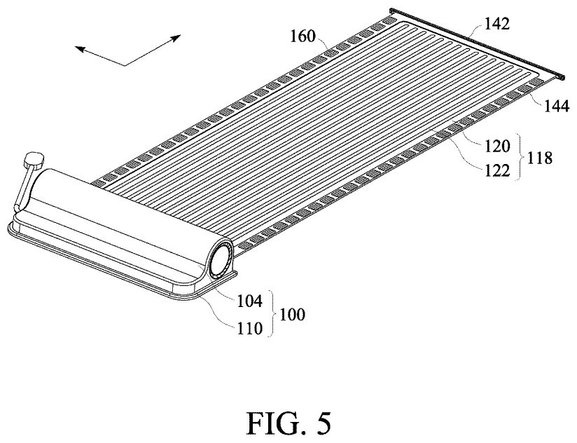

Referring to FIGS. 2 and 5, in some embodiments, the flexible light board further includes an integrated solar panel 160 configured to recharge the internal power supply by converting ambient sunlight into electrical energy. The solar panel 160 is mounted on the flexible pad between longitudinal light strip 126 and flexible wear-resistant strip 144, enabling direct exposure to sunlight during outdoor use or storage. The solar panel 160 is electrically connected to an energy storage element 162, further connected to the circuit board 158, which regulates charging current and voltage to protect internal circuits and prevent overcharging. The addition of the solar panel 160 allows the flexible light board to be recharged without reliance on external power sources, thereby enhancing portability and convenience for the user. This configuration is especially suitable for field use, travel, or environments where electrical outlets are not readily available. Optionally, the solar panel includes a protective transparent layer formed of high-strength, scratch-resistant material to ensure long-term durability.

The solar panel 160 is formed using and not limiting, alone or in combination of photovoltaic material monocrystalline silicon, polycrystalline silicon, thin-film amorphous silicon, copper indium gallium selenide (CIGS), organic photovoltaic films, perovskite photovoltaic layers, or flexible composite photovoltaic laminates, capable of converting sunlight into electrical energy. The solar panel 160 further includes a transparent protective cover formed of tempered glass, polycarbonate, acrylic, or any impact-resistant optical polymer to improve durability and light transmission. The electrical energy generated by the solar panel 160 is stored in the energy storage element 162, placed near, such as a rechargeable lithium-ion battery, lithium-polymer battery, nickel-metal hydride (NiMH) cell, thin-film solid-state battery, supercapacitor module, or a combination of multiple storage components. The type, capacity, and arrangement of the energy storage element 162 are not limited and may be selected according to desired device performance.

In some embodiments, the energy storage element 162 is electrically coupled to the circuit board 158, within which a charging management circuit is provided to regulate charging current, prevent overcharging, balance cell voltage, and manage charging from both the solar panel 160 and any external power source.

In some embodiments, the circuit board 158 is in the storage cavity, and the longitudinal light strip 126 is directly connected to the transverse light strip 124, which shortens the wiring distance and simplifies the wiring arrangement. Specifically, each longitudinal light strip 126 extends along the length direction or X-direction of the flexible pad 120, ensuring that the overall length and width of the flexible pad 120 can provide a uniform illumination effect. This configuration also facilitates storage, as the longitudinal light strips 126 can be uniformly wound onto the rotating shaft 128.

Referring to FIGS. 3 and 4, in some embodiments, the flexible phototherapy device further includes the circuit board 158 and a conductive interface. The conductive interface includes a conductive disk 146. The circuit board 158 is fixedly installed in the storage cavity and is electrically connected to both the driving member 132 and the conductive disk 146. The conductive disk 146 is also installed in the storage cavity in a relatively fixed position. The proximal end of the flexible light board 118 is mounted on the rotating shaft 128, and its position is relatively stable. The conductive disk 146 is electrically connected to the proximal end of the flexible light board 118, ensuring stable electrical contact during storage, unfolding, and use, and preventing electrical disconnection caused by movement or deformation of the flexible light board 118.

In one embodiment, again with reference to FIGS. 3 and 4, the conductive disk 146 includes a mounting disk 148 and a rotating disk 150. The mounting disk 148 is installed on the storage seat 100, while the rotating disk 150 is installed on one end of the rotating shaft 128 and is connected to the flexible light board 118. Since the first end of the flexible light board 118 is fixed to the rotating shaft 128, attaching the rotating disk 150 to the end of the rotating shaft 128 does not interfere with the flexible light board 118, and facilitates electrical installation at the first end of the flexible light board 118, improving wiring and connection reliability.

In one embodiment, alone or in combination, the mounting disk 148 and the rotating disk 150 are provided with a conductive ring 152, and the other is equipped with a conductive spring pin 154. The conductive spring pin 154 presses against the conductive ring 152 under elastic force, enabling it to accommodate slight deformation or positional deviation of the conductive ring 152. This reduces the likelihood of circuit failure caused by poor contact and ensures a reliable and stable electrical connection between the two, unaffected by external disturbances.

In some embodiments, the conductive ring 152 and the rotating shaft 128 are coaxially arranged to ensure stable electrical contact during relative rotation. Multiple conductive spring pins 154 are arranged corresponding to multiple concentric conductive rings 152, with each conductive spring pin 154 elastically contacting the respective conductive ring 152. The concentric arrangement of multiple conductive rings 152 enables multiple independent electrical channels for power and/or signal transmission between the stationary mounting disk 148 and the rotating disk 150. The rotating disk 150 is provided with the conductive spring pins 154, and the mounting disk 148 is provided with the conductive ring 152. The mounting disk 148 and the rotating disk 150 are rotatably connected to each other, ensuring positional stability and reliable power transmission between them. The rotating disk 150 is positioned relative to the rotating shaft 128 using a positioning structure, eliminating the need for screws, adhesives, or welding. This simplifies assembly and allows the rotating disk 150 to be quickly and accurately connected to the rotating shaft 128, improving assembly efficiency. The rotating shaft 128 is provided with a positioning protrusion 130, and the rotating disk 150 is provided with a first positioning slot 156. The positioning protrusion 130 is received within the first positioning slot 156, thereby enabling the rotating disk 150 to be positioned relative to the rotating shaft 128 and rotate synchronously. The rotating shaft 128 is formed as a sleeve by stamping, with the positioning protrusion 130 formed on its inner or outer surface as needed. The outer surface of the rotating shaft 128 may include surface features such as texturing, ridges, knurling, or other formations to increase the connection area with the first end of the flexible light board 118, facilitating stronger attachment. The first end of the flexible light board 118 is connected to the rotating shaft 128 by bonding, fasteners, welding, clamping, etc. When the rotating shaft 128 is formed as a stamped sleeve, its outer surface is shaped to increase the connection area with the first end of the flexible light board 118, facilitating strong attachment, including fastener-based attachment.

Additionally, the rotating shaft 128, the mounting disk 148, and the rotating disk 150 are arranged coaxially, allowing them to be compactly located within the device. This helps reduce the space occupied by the flexible phototherapy device. Their coaxial arrangement also improves positional stability and contributes to reliable and stable power transmission.

Referring to FIGS. 2 and 4, in some embodiments, the driving member 132 is received within the rotating shaft 128. One end of the driving member 132 is exposed and connected to the storage seat 100, while the other end includes a rotatable output shaft 134. The rotatable output shaft 134 extends into the rotating shaft 128 and is connected thereto. By allowing the rotatable output shaft 134 to extend into the rotating shaft 128, the external space required for the drive structure is significantly reduced, which is beneficial for compact device design. Furthermore, the rotatable output shaft 134 directly drives the rotating shaft 128, which reduces the number of intermediate transmission components, lowers costs, and further reduces the device's overall space requirements.

In one embodiment, the flexible phototherapy device further includes a rotation transmission block 136. The rotation transmission block 136 is fixedly sleeved on the rotatable output shaft 134, and the rotation transmission block 136 is sleeved on the rotating shaft 128 for internal rotation. The rotation transmission block 136 has a larger size than the rotatable output shaft 134, thereby increasing the transmission contact area with the rotating shaft 128, which improves transmission efficiency and operational stability.

The rotation transmission block 136 is positioned relative to the rotating shaft 128 through a positioning structure, such that transmission between the two is achieved without requiring screw fastening, bonding, or welding. This simplifies connection operations, and the rotation transmission block 136 can be quickly and accurately connected, thereby improving assembly efficiency. The rotating shaft 128 includes the positioning protrusion 130, and the rotation transmission block 136 includes a second positioning groove 140, wherein the positioning protrusion 130 is embedded within the second positioning groove 140 to achieve synchronous rotation between the components.

In another embodiment, the flexible phototherapy device further includes a rotating support block 138. The rotating support block 138 is rotatably sleeved on the driving member 132, and the rotating support block 138 is also sleeved on the rotating shaft 128. The rotating support block 138 rotates synchronously with the rotating shaft 128, and receives support from the driving member 132, thereby stabilizing the rotation of the rotating shaft 128 and preventing deviation in its rotational position, which increases the storage stability of the flexible light board 118.

The rotating support block 138 is positioned relative to the rotating shaft 128 through a positioning structure, enabling transmission between the two without the need for screw fastening, bonding, or welding, which simplifies assembly operations. The rotation transmission block 136 can therefore be quickly and accurately connected, further improving assembly efficiency. Optionally, the rotating shaft 128 includes the positioning protrusion 130, and the rotating support block 138 includes a third positioning groove, into which the positioning protrusion 130 is embedded to achieve synchronous rotation of both components. The third positioning groove of the rotating support block 138 engages with the positioning protrusion 130, like the first positioning slot 156 of the rotating disk 150 and the second positioning groove 140 of the rotation transmission block 136. The rotating support block 138 and the rotation transmission block 136 are located at opposite ends of the driving member 132, thereby providing stable axial support for the rotating shaft 128 during rotation. The driving member 132 may be a motor, a rotary cylinder, or a rotary oil cylinder, although not limited to these examples.

Referring to FIG. 3 and FIG. 5, in some embodiments, the storage seat 100 includes a first housing 104 connected to a second housing 110, wherein the first housing 104 and the second housing 110 collectively form an enclosed storage cavity. The connection arrangement between the first housing 104 and the second housing 110 allows easy installation and removal by the user, while enabling convenient maintenance of the internal structure for long-term reliability.

Referring to FIG. 2 and FIG. 4, in one embodiment, the first housing 104 includes a first connecting hole 106, and the second housing 110 includes a second connecting hole 112 corresponding to the first connecting hole 106. A fastener passes through the first connecting hole 106 and the second connecting hole 112 to tightly secure the first housing 104 and the second housing 110, thereby enhancing the structural strength and stability of the storage seat 100, making it more durable. Further, a sealing strip 116 is provided between the first housing 104 and the second housing 110, which offers favourable sealing performance such that the storage seat 100 protects internal components from external unfavourable physical conditions as wear, moisture, and dust.

In another embodiment, the first housing 104 includes a socket 108, and the second housing 110 includes an insert 114 (also referred to as an insertion structure), wherein the insert 114 is removably inserted into the socket 108. The plugin engagement between the insert 114 and the socket 108 enables the first housing 104 and the second housing 110 to connect quickly without additional fasteners or tools. After the insert 114 is inserted into the socket 108, a snap or locking effect may be formed, enhancing the stability and fastening strength of the storage seat 100.

In another embodiment, the first connecting hole 106 and the second connecting hole 112 are aligned along a first direction, and the insert 114 (insertion structure) is detachably inserted into the socket 108 along a second direction. The angle between the first direction and the second direction is between 60° to 90°. Arranging the connections between the first housing 104 and the second housing 110 in different first and second directions creates a cross-fixing effect, thereby enhancing overall stability and reducing the likelihood of loosening due to vibration or external forces.

In an embodiment, after the insert 114 is detachably inserted into the socket 108 along the second direction, the relative positions of the first housing 104 and the second housing 110 remain substantially fixed without shaking, which facilitates inserting a fastener through the first connecting hole 106 and the second connecting hole 112. Optionally, the first direction and second direction are perpendicular, with the angle between them being 90°, further enhancing the cross-fixing effect. The first direction corresponds to the thickness direction of the phototherapy cap, while the second direction corresponds to the length direction of the unfolded phototherapy cap.

The embodiments described above collectively demonstrate a flexible phototherapy device featuring a compact, self-retracting light board mechanism that enables convenient storage, enhanced portability, and improved protection of the flexible light strip. Through the cooperation of the rotating shaft, internal motorized driving member, limiting structures, flexible wear-resistant strip, and reinforced shaft-coupling members, the device achieves stable mechanical operation during repeated winding and unwinding cycles. The flexible pad with its multi-flexible light strip array provides wide and uniform illumination, while the integrated protective flexible pad structure shields the flexible light strips from abrasion and mechanical shock, ensuring long-term reliability in both clinical and home environments.

In some embodiments, a method for deploying and retracting a flexible phototherapy light board is provided. The method comprises coupling a proximal end of a flexible light board 118 to a rotating shaft 128 positioned within a storage seat 100. The rotating shaft 128 is rotated in a first direction with a driving member 132 to wind the flexible light board 118 around the rotating shaft 128 and retract the flexible light board 118 into a storage cavity of the storage seat 100. The flexible light board 118 is extended from the storage cavity by rotating the rotating shaft 128 in a second, opposite direction or by pulling the flexible light board 118 outward through a storage port 102. Rotating the rotating shaft 128 in the first direction comprises driving a rotatable output shaft 134 disposed within the rotating shaft 128. Electrical conductivity between the flexible light board 118 and a circuit board 158 is maintained through a conductive interface while the rotating shaft 128 rotates. The conductive interface includes a mounting disk 148 and a rotating disk 150 with conductive rings 152 and conductive spring pins 154 that maintain electrical contact during rotation. Complete retraction of the flexible light board 118 is prevented by engaging a limiting member 142 at its distal end with an edge of the storage port 102. The limiting member 142 has a width greater than the width of the storage port 102, such that the limiting member 142 cannot pass through the storage port 102 into the storage cavity, thereby ensuring the flexible light board 118 remains accessible for subsequent deployment.

In some embodiments, the flexible phototherapy device may be configured for use as a window curtain, allowing the user to receive therapy while optimizing space. In this configuration, the storage seat may be mounted above a window or door frame using mounting brackets, hooks, rails, or other suitable fastening mechanisms. The mounting brackets may be configured to attach to the wall surface above the window frame, to the ceiling adjacent to the window, or directly to the window frame itself. The storage seat may include mounting holes or slots that align with corresponding fasteners on the mounting brackets. When installed, the flexible light board extends downward from the storage seat through the storage port in a curtain-like manner, hanging vertically in front of the window or door opening. The driving member may be operated to control the extent of deployment, allowing the user to adjust how much of the flexible light board is extended. This arrangement enables the user to receive phototherapy even when the window remains closed, such as during winter months when opening the window is not desirable due to cold temperatures. The curtain configuration integrates the therapeutic function into the living environment without requiring dedicated floor space or additional furniture, thereby optimizing the use of available space in the room. The flexible light board may be retracted fully into the storage seat when not in use, providing an unobstructed view through the window. The curtain configuration may also complement existing window treatments and home decor, as the storage seat and flexible light board may be designed in various colors, finishes, or materials to match the interior design of the room.

In some embodiments, the flexible phototherapy device may function as a foldable panel. In this configuration, the storage seat rests on the floor, or a supporting surface such as a table, desk, or countertop, and the user raises the flexible light board to an upright position to receive therapy. The flexible light board may be extended from the storage seat and positioned at an angle relative to the floor or supporting surface, with the light-emitting surface facing the user. After use, the flexible light board can be folded down and retracted into the storage seat for compact storage. A support stick, stand, or frame may be provided to hold the flexible light board in an upright position during operation, ensuring stable positioning throughout the therapy session. The support stick may be a telescoping rod, a hinged arm, or a rigid strut that connects between the storage seat and a point along the flexible light board or the limiting member at the distal end. The support stick may include an adjustable joint or pivot that allows the user to set the angle of the flexible light board, enabling the user to direct the therapeutic light toward different body areas such as the face, neck, chest, or hands. In some cases, the support stick may be detachable and stored within or alongside the storage seat when not in use. The foldable panel configuration allows the user to position themselves in front of the extended flexible light board, such as by sitting in a chair or standing, to receive phototherapy on exposed skin areas. The adjustable angle feature enables customization of the therapy session based on the user's height, seating position, or target treatment area.

In some embodiments, the rear side of the flexible light board, opposite the light-emitting surface, may incorporate a flexible solar sheet. The flexible solar sheet is positioned on the back surface of the flexible pad, between the flexible pad material and any protective outer layer. The flexible solar sheet may be formed using flexible photovoltaic materials such as amorphous silicon, copper indium gallium selenide (CIGS), organic photovoltaic films, perovskite photovoltaic layers, or other thin-film solar cell technologies capable of bending without damage. The flexible solar sheet is electrically connected to the energy storage element through wiring that runs along the flexible light board and through the conductive interface at the rotating shaft. A charge controller or charging management circuit on the circuit board regulates the current and voltage from the flexible solar sheet to safely charge the energy storage element. The flexible solar sheet may include a transparent protective layer formed of a flexible polymer material such as ethylene tetrafluoroethylene (ETFE), polyethylene terephthalate (PET), or a similar scratch-resistant and weather-resistant material to protect the photovoltaic cells from abrasion and environmental exposure. The stored energy can then be used to power the LEDs while the device is mounted on a window or door, enabling cordless operation. This configuration is particularly advantageous when the device is used in the window curtain arrangement, as the rear-facing solar sheet can continuously collect sunlight through the window while the front-facing LEDs deliver therapy to the user. The dual functionality of the window curtain configuration allows simultaneous energy harvesting and therapy delivery, extending the operational time of the device without requiring connection to an external power source.

In some embodiments, the flexible phototherapy device may include a sensor or detection mechanism configured to automatically illuminate the LEDs upon detecting that the flexible light board is unfolded or extended from the storage cavity. The sensor may be a position sensor, a Hall effect sensor, a limit switch, an optical sensor, a rotary encoder, or any other suitable sensing device capable of detecting the rotational position of the rotating shaft or the linear extension of the flexible light board through the storage port. The sensor may be mounted within the storage cavity and electrically connected to the circuit board or control module. When the sensor detects that the flexible light board has been extended beyond a predetermined threshold, the control module may automatically activate the LEDs to begin the therapy session. Alternatively, only the LEDs corresponding to the exposed or unfolded area of the flexible light board may be activated, while LEDs in the still-wound portion remain off. This zone-based illumination control may be achieved by dividing the flexible light strip into multiple independently controllable segments or zones, each connected to the circuit board through separate control lines or addressable LED drivers. The control module may determine the degree of unfolding based on the sensor input and activate only the LED zones that are exposed outside the storage cavity. This selective illumination conserves energy and prevents unnecessary heat generation in the stored portion of the flexible light board. The automatic illumination feature enhances user convenience by eliminating the need to manually activate the LEDs after extending the flexible light board, and the zone-based control optimizes energy efficiency during partial deployment scenarios.

In some embodiments, the flexible phototherapy device may include an IR sensor (infrared sensor) configured to detect whether a user is present in front of the flexible light board. The IR sensor may be mounted on the storage seat, the top bar, or the flexible light board itself. When the IR sensor detects user presence, the control module automatically activates the LEDs to begin the therapy session. Conversely, when the user moves away and is no longer detected by the IR sensor, the control module automatically deactivates the LEDs to conserve energy. The IR sensor may be a passive infrared (PIR) sensor, an active infrared sensor, or other suitable proximity detection sensor capable of detecting human presence. This automatic on/off feature enhances user convenience by eliminating the need for manual activation and improves energy efficiency by ensuring the LEDs operate only when a user is positioned to receive therapy. The detection range and sensitivity of the IR sensor may be adjustable to accommodate different room configurations and user preferences.

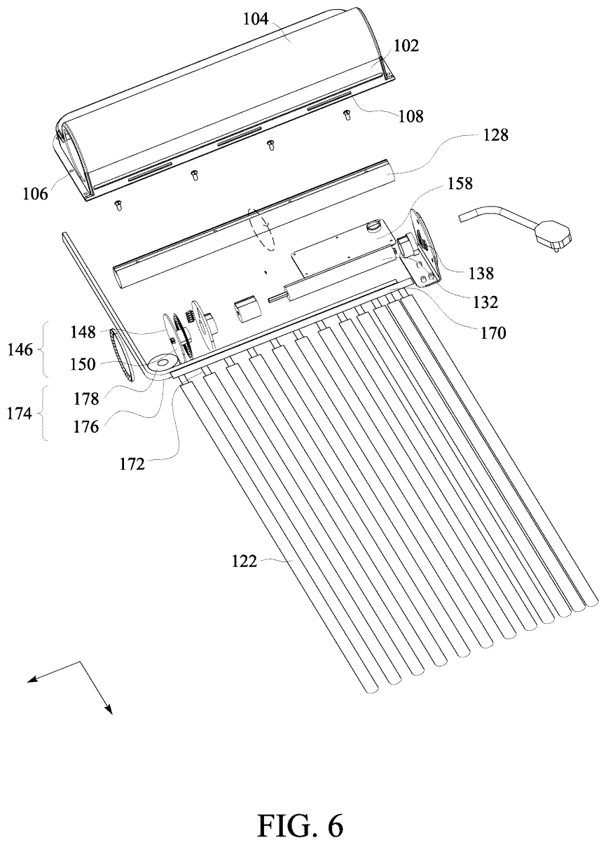

In some embodiments, and referring to FIG. 6, two or more flexible light boards may be arranged in a horizontal sliding channel configuration, similar to horizontal blinds or sliding panel curtains. In this arrangement, a top bar 170 is fixed to and extends from the storage seat 100 at the storage port side. The top bar 170 extends along the length direction of the storage seat 100. The storage seat 100 houses the rotating shaft 128 and the driving member 132. A motion conversion mechanism 174 converts rotational movement from the rotating shaft 128 into linear horizontal movement. The motion conversion mechanism 174 may include a pinion gear 178 coupled to the rotating shaft 128 or the rotatable output shaft 134, and the pinion gear 178 engages a rack 176 extending along the length of the top bar 170, such that rotation of the pinion gear 178 causes linear movement along the rack 176. Alternatively, the motion conversion mechanism 174 may include a belt drive system, a lead screw assembly, a cable drum mechanism, or other suitable gear or transmission systems capable of translating rotational motion into linear translation. Each flexible light board 118 is suspended from the top bar 170 via sliding carriers 172, rollers, hooks, or other suitable sliding mechanisms that allow the flexible light boards 118 to move along the length of the channel. Unlike the wound storage configuration, in this horizontal sliding arrangement, the flexible light boards 118 are not retracted into the storage cavity by winding around the rotating shaft 128; rather, the flexible light boards 118 remain in their extended flat form and are moved horizontally along the channel by the converted linear motion from the rotating shaft 128. When deployed, the flexible light boards 118 slide outward from one end of the channel to cover the window or door opening, with adjacent flexible light boards 118 positioned side-by-side to provide continuous or overlapping coverage across the width of the opening. When retracted, the flexible light boards 118 slide horizontally toward the storage seat 100 end of the channel, stacking or gathering at that end to clear the window or door opening. The top bar 170 may include a track, groove, or rail structure that guides the sliding movement of the flexible light boards 118 and maintains their vertical orientation during horizontal translation. Each flexible light board 118 may be independently slidable, allowing the user to selectively position individual boards to cover specific portions of the window opening, or the flexible light boards 118 may be linked together so that moving one board causes adjacent boards to follow in sequence. The conductive interface, including the mounting disk 148 and rotating disk 150 with conductive rings 152 and conductive spring pins 154 as described in the preceding embodiments, may be used to provide electrical power from the circuit board 158 to the flexible light boards 118 through the rotating shaft 128 and subsequently through flexible wiring harnesses or sliding electrical contacts along the top bar 170. A control module may be provided to operate the driving member 132 for automated deployment and retraction of the flexible light boards 118 along the channel via the motion conversion mechanism 174. Each flexible light board 118 may be independently controlled for illumination, allowing the user to activate LEDs on selected boards while leaving others inactive. For example, if only partial coverage is desired, the user may deploy and illuminate only one or two flexible light boards 118 while the remaining boards stay gathered at the end of the channel. This horizontal sliding channel configuration is particularly suitable for covering large window or door openings where a single flexible light board 118 would be insufficient, and the side-by-side arrangement of multiple boards enables scalable coverage for windows of varying widths. The configuration also allows for selective therapy delivery, as the user can position themselves adjacent to specific illuminated boards to target particular body areas.

The invention further incorporates a solar-assisted power system in which a flexible photovoltaic panel and an onboard energy-storage element cooperate with a charging-management circuit to permit cable-free operation for extended durations. This configuration enables the device to be used in resource-limited settings, travel situations, outdoor environments, and other locations where external power sources are unavailable. The slip-ring conductive interface and internally mounted circuit board maintain continuous electrical connectivity regardless of the rotating shaft's rotation, thereby supporting consistent illumination performance. By integrating the mechanical winding system with the power supply and LED-driving architecture, the device provides a unified and highly portable phototherapy platform.

In some embodiments, the flexible phototherapy device may be configured for floor-based use. In this configuration, the storage seat rests on the floor or is supported by a floor stand, and the flexible light board extends upward or outward from the storage seat. The floor stand may include a weighted base, adjustable legs, or caster wheels for stability and mobility. The flexible light board may be extended vertically to form a standing panel, allowing the user to stand or sit in front of the device to receive therapy on the face, torso, or full body. Alternatively, the flexible light board may be extended horizontally or at an inclined angle to provide therapy to a user lying on the floor, a mat, or a bed positioned adjacent to the device. The driving member may be operated to adjust the extent of deployment, and the support stick or an integrated frame may hold the flexible light board at a desired angle. This floor-based configuration is suitable for full-body phototherapy sessions, yoga or meditation environments, home wellness spaces, or clinical treatment rooms where wall or window mounting is not practical.

In some embodiments, the flexible phototherapy device may be configured for wall-mounted use. In this configuration, the storage seat is mounted on a wall surface using mounting brackets, screws, anchors, or other suitable fastening mechanisms. The mounting brackets may be configured to attach the storage seat at various heights on the wall, allowing the user to position the device at a level suitable for treating the face, upper body, or lower body. When installed, the flexible light board extends outward from the storage seat through the storage port, either hanging downward in a curtain-like manner or projecting horizontally from the wall. The flexible light board may be held in a horizontal or angled position by a support arm, hinged bracket, or tensioned cable attached to the wall or ceiling. The driving member may be operated to deploy or retract the flexible light board as needed. This wall-mounted configuration saves floor space and integrates the phototherapy device into the room environment, making it suitable for bathrooms, bedrooms, home gyms, spa treatment rooms, or clinical settings where floor space is limited.

In some embodiments, the flexible phototherapy device may include a universal mounting system that allows the storage seat to be installed on either a floor stand or a wall bracket without modification. The storage seat may include mounting holes, slots, or attachment points on multiple surfaces, enabling the user to select the preferred installation orientation. A quick-release mechanism or tool-free fastening system may be provided to allow the user to switch between floor and wall configurations. This versatility enables the device to be repositioned based on the treatment area, room layout, or user preference, and allows a single device to serve multiple use cases in different environments.

The invention finds industrial application in the cosmetics, dermatology, personal care, and wellness device industries, where compact, multi-modal treatment tools are in high demand. The device may be mass-produced using conventional plastic moulding, electronic assembly, and consumer-grade manufacturing processes, making it suitable for large-scale commercial production. It may be marketed as a premium home-use skincare tool, professional aesthetician device, or integrated therapy instrument for salons, spas, dermatology clinics, and beauty centers. Its ergonomic design, modular stimulation components, and compatibility with skincare product containers offer significant advantages for both consumer and professional markets.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A flexible phototherapy device, comprising:
a storage seat defining a storage cavity and a storage port communicating with the storage cavity;
a flexible light board having a proximal end and a distal end, the proximal end being fixed to a rotating shaft that is rotatably supported within the storage cavity, the distal end being extendable through the storage port;
a driving member operatively coupled to the rotating shaft and configured to rotate the rotating shaft to wind the flexible light board around the rotating shaft for retraction of the flexible light board into the storage cavity; and
a conductive interface located in the storage cavity and electrically connected to the proximal end of the flexible light board to provide electrical power to the flexible light board during extension and retraction.

2. The device of claim 1, wherein the distal end of the flexible light board includes a limiting member having a width greater than width of the storage port, the limiting member preventing full retraction of the distal end into the storage cavity.

3. The device of claim 1, further comprising a flexible wear-resistant strip disposed on at least one side of the flexible light board and configured to reduce abrasion between the flexible light board and the storage port.

4. The device of claim 1, wherein the flexible light board comprises a flexible pad and a flexible light strip embedded in the flexible pad.

5. The device of claim 4, wherein the flexible light strip comprises a transverse light strip extending across a width of the flexible pad and multiple longitudinal light strips connected to the transverse light strip and extending along a length of the flexible pad.

6. The device of claim 1, wherein the conductive interface comprises a fixed conductive member electrically connected to a circuit board in the storage cavity and a movable conductive member coupled to the rotating shaft.

7. The device of claim 6, wherein the fixed conductive member comprises a conductive ring and the movable conductive member comprises a conductive spring pin in sliding contact with the conductive ring.

8. The device of claim 1, wherein the driving member has a rotatable output shaft extending into the interior of the rotating shaft and coupled thereto.

9. The device of claim 8, further comprising a rotation transmission block secured to the rotatable output shaft and positioned inside the rotating shaft to transmit rotational force to the rotating shaft.

10. The device of claim 1, wherein the storage seat comprises a first housing and a second housing joined together, the first housing including a connecting hole aligned with a connecting hole of the second housing, and an insertion structure extending into a seam between the first housing and the second housing.

11. The device of claim 1, wherein the storage seat is configured for mounting above a window or door frame such that the flexible light board extends downward through the storage port, or wherein the device further comprises a support stick configured to hold the flexible light board in an upright position when the storage seat rests on a supporting surface.

12. The device of claim 1, wherein the flexible light board includes a flexible solar sheet on a rear side opposite a light-emitting surface, the flexible solar sheet being electrically connected to an energy storage element.

13. The device of claim 1, further comprising an energy storage element and a solar panel electrically connected to the energy storage element and configured to recharge the energy storage element using ambient light.

14. A method for deploying and retracting a flexible phototherapy light board, comprising:
coupling a proximal end of a flexible light board to a rotating shaft positioned within a storage seat;
rotating the rotating shaft in a first direction with a driving member to wind the flexible light board around the rotating shaft and retract the flexible light board into a storage cavity of the storage seat; and
extending the flexible light board from the storage cavity by rotating the rotating shaft in a second, opposite direction, or by pulling the flexible light board outward through a storage port.

15. The method of claim 14, wherein rotating the rotating shaft in the first direction comprises driving a rotatable output shaft disposed within the rotating shaft.

16. The method of claim 14, further comprising maintaining electrical conductivity between the flexible light board and a circuit board through a conductive interface while the rotating shaft rotates.

17. A flexible phototherapy device, comprising:
a storage seat defining a storage cavity and a storage port communicating with the storage cavity, the storage seat configured to span across the width of a window;
a top bar fixed to and extending from the storage seat at the storage port side, the top bar extending along a length direction of the storage seat;
a flexible light board suspended from the top bar via sliding carriers and configured to slide horizontally along the top bar;
a rotating shaft rotatably supported within the storage cavity;
a driving member operatively coupled to the rotating shaft;
a motion conversion mechanism configured to convert rotational movement of the rotating shaft into linear horizontal movement along the top bar; and
a conductive interface located in the storage cavity and electrically connected to the flexible light board to provide electrical power to the flexible light board.

18. The device of claim 17, wherein the motion conversion mechanism includes a pinion gear coupled to the rotating shaft and a rack extending along the top bar, the pinion gear engaging the rack.

19. The device of claim 17, wherein two or more flexible light boards are suspended from the top bar and configured to slide horizontally along the top bar, the flexible light boards configured to stack at one end of the top bar when retracted and to slide outward to cover a window or door opening when deployed.

20. The device of claim 19, wherein each flexible light board is independently controllable for illumination.
